(19) United States
(12) Patent Application Publication
(10) Pub. No.: US 2024/0032493 A1
(43) Pub. Date: Feb. 1, 2024

(54) SYSTEMS AND METHODS RELATING TO PROTOCOLS IN PLANT BREEDING PIPELINES

(71) Applicant: MONSANTO TECHNOLOGY LLC, Saint Louis, MO (US)

(72) Inventors: Xin SHEN, Mountain View, CA (US); Aviral SHUKLA, Defiance, MO (US); Slobodan TRIFUNOVIC, Wildwood, MO (US); Yiduo ZHAN, Chesterfield, MO (US); Zihao ZHAO, Woodinville, WA (US)

(21) Appl. No.: 18/028,173

(22) PCT Filed: Sep. 17, 2021
(86) PCT No.: PCT/US2021/050996

§ 371 Date: Mar. 23, 2023

Related U.S. Application Data
(60) Provisional application No. 63/082,952, filed on Sep. 24, 2020.

(51) Int. Cl. A01H 1/02; A01H 1/04

(52) U.S. Cl. CPC A01H 1/02; A01H 1/04

(57) ABSTRACT

Systems and methods are provided for automatically allocating test protocols to a plurality of test locations. Once such method includes a computing device executing a first stage machine learning prediction model (MLPM) based on protocol data for multiple test protocols for a test experiment to generate a first stage output. The first stage MLPM is trained based on historical allocation data for one or more prior test experiments. Multiple test sets are associated with the test protocols, and the first stage output includes, for multiple test locations, allocation prediction scores for the test protocols. Based on the first stage output, the computing device executes a second stage optimization model to generate a second stage output. The second stage output includes an allocation plan for the test protocols. The allocation plan identifies one or more of the test locations for each of the test protocols.

### CROSS-REFERENCE TO RELATED APPLICATION

This application claims the benefit of, and priority to, U.S. Provisional Patent Application No. 63/082,952, filed Sep. 24, 2020, the entire disclosure of which is incorporated herein by reference.

### FIELD

The present disclosure generally relates to systems and methods for use with a plant breeding pipeline to allocate protocols (e.g., test protocols, etc.) associated with the plant breeding pipeline to locations (e.g., test locations, etc.) within a network of locations.

### BACKGROUND

This section provides background information related to the present disclosure which is not necessarily prior art.

In plant development, modifications are often made in plants either through selective breeding or genetic manipulation. Based on the particular selection or manipulation, the resulting plant material (e.g., hybrid seeds, etc.) is introduced into a breeding pipeline, where plants are then created, grown, and tested. In connection with such selection or manipulation, environmental features associated with differences in yield, standability, disease, etc. for hybrid plants are often taken into account (e.g., different soil types, climatic and edaphic conditions, crop-years, etc.). To determine suitable environments for different hybrid plants, experiments are performed as the seeds/plants advance through the breeding pipeline. In so doing, different hybrid seeds/plants are grouped into test sets, with groups of the different test sets each associated with different test protocols. Each test protocol, then, includes one or more parameters for testing the seeds/plants of the test sets associated with the test protocols. The parameters for each test protocol generally dictate the requirements for testing the given seeds/plants, as well as characteristics of the seeds/plants and the test sets associated with the test protocol, such that seeds/plants that are in test sets that have been grouped into the same protocol are to generally be tested in common or coordinated environments.

### DRAWINGS

The drawings described herein are for illustrative purposes of selected embodiments and are not intended to limit the scope of the present disclosure.

FIG. 1 is a block diagram of an example system of the present disclosure suitable for use in automatically allocating protocols (e.g., of a test experiment, etc.) to locations within a network of locations;

FIG. 2 is a block diagram of a computing device that may be used in the example system of FIG. 1;

FIG. 3 illustrates an example map that provides a visualization based on an output associated with a first stage of the system of FIG. 1;

FIG. 4 illustrates an example user interface that may be generated by the system of FIG. 1; and

FIG. 5 is an example method, suitable for use with the system of FIG. 1, for automatically allocating protocols (e.g., of a test experiment, etc.) to locations within a network of locations.

Corresponding reference numerals indicate corresponding parts throughout the several views of the drawings.

### DETAILED DESCRIPTION

Example embodiments will now be described more fully with reference to the accompanying drawings. The description and specific examples included herein are intended for purposes of illustration only and are not intended to limit the scope of the present disclosure.

To determine the desired environments for different hybrid plants (e.g., environments that will produce acceptable or desired yields, etc.), test experiments are performed as the seeds/plants advance through a breeding pipeline. In connection therewith, different hybrid seeds (e.g., hybrid soy seeds, hybrid corn seeds, etc.) are grouped into test sets based on one or more characteristics such as relative maturity (e.g., thousands of test sets each having 60, 120, 240, etc. different hybrid seeds), etc. Groups of the different test sets are each associated with or assigned to a different one of a plurality of test protocols (e.g., several hundred test protocols each having 10 grouped test sets assigned thereto, etc.). Each test protocol includes test protocol data. The test protocol data generally dictates the requirements for testing the seeds of the test sets assigned to the test protocols, as well as characteristics of the seeds of the test sets assigned to the test protocols.

To conduct the test experiments, the test sets of the test protocols may be manually assigned by an individual to different hubs within a network of locations (broadly, test locations). Each region includes a hub, where test locations within the region are assigned to that hub. Personnel associated with the hub, then, are generally responsible for growing the test sets assigned to the different test locations within the corresponding region (but not to test locations that are assigned to other hubs (e.g., to test locations out of the given region, etc.)).

The test locations may include, for example, outdoor field sites, indoor grow sites, etc. At any given time, each test location is associated with one or more characteristics such as, for example, a geographic location (e.g., a latitude and/or longitude, etc.), a region (e.g., an eastern, western, northern, or southern region of United States, etc.), a season (e.g., a summer, spring, fall, winter season, etc.), a size (e.g., a number of acres, a number of plots, etc.), a capacity (e.g., a maximum usable number of acres, etc.), a stage (e.g., early, mid, or late stage planting, etc.), plant type(s) (e.g., soy and/or corn hybrid plants, etc.), staffing (e.g., a number of testers, etc.), macro-environments (MACs) (e.g., MAC6.6, etc.), soil type(s) (e.g., s1727, etc.), product segment(s) (e.g., Central, Delta , etc.), special rules (e.g., a rule that test protocols with maturity of −0.9 should not be allocated to locations in the United States, etc.), maturity (e.g., relative maturity of seeds/plants growing at the location, etc.), etc.

In manually assigning the test sets to the different hubs, the individual attempts to ensure that the test requirements are satisfied and that the environments of the test sites assigned to the hubs accommodate the characteristics of the test sets. This approach is problematic, though. For instance, a test experiment may have a number Nₜₚ of test protocols and a number Nₜₗ of test locations. In connection therewith, the number Nₐₚ of possible allocation plans for the test protocols is 2^{N}^{_{tP}}^{×N}^{ₜₗ}. For example, the number Nₜₚ of test protocols for a given test experiment may be 1000 or more, and the number Nₜₗ of test locations may, for example, be 800 or more, resulting in a number Nₜ of possible allocation plans of approximately 10²⁴⁰⁸²³. Practically speaking, it is impossible for an individual to manually allocate test sets for these test protocols among the test locations, while taking into account the parameters of the test protocol and the characteristics of the test locations (e.g., test location capacity, region balance, co-location preferences between different test stages, etc.), while also making sure that each test set associated with the experiment is advanced and grown to a suitable test location.

What's more, each hub functions independently within the network, while the parameters of a given test protocol (for which test sets may be advanced to different hubs within the network) remain the same for all of the test sets of the test protocol. As a result, parameters of the test protocol may be inappropriately applied by the independent hubs advancing different test sets of the same test protocol to different locations in different regions. For example, a test protocol may require testing to be split evenly into two different product segments, where the two different product segments are associated with, or belong to, four different hubs. If one associated hub allocates the test protocol to three test locations in the first product segment and five test locations in the second product segment, the other three hubs will need to adjust their allocation accordingly to make sure the overall allocated locations are evenly split between the two product segments, yet there is only manual interaction among those hubs.

Uniquely, the systems and methods herein provide for use of artificial intelligence in a breeding pipeline to automatically allocate protocols (e.g., test protocols of a test experiment, other protocols, etc.) to locations (e.g., test locations, other locations, etc.) within a network of locations, given the hubs to which test locations may be assigned, the number of locations within the network, and the number of protocols or sets of the test experiment, etc. In one particular embodiment, an intelligence engine is configured to receive parameters for testing a plurality of seeds of a plurality of test sets associated with a plurality of test protocols. The intelligence engine is configured to, based on the received parameters and a first stage machine learning prediction model (MLPM), automatically generate a probability matrix indicating probabilities that the test locations satisfy the parameters for the test protocols. In so doing, the probabilities are based on historical allocation data used to train the first stage MLPM. The intelligence engine, then, is also configured to subject a second stage optimization model (OM) to a plurality of constraints and to, based on the probability matrix and the second stage OM, automatically generate allocation plans for the test experiment. The allocation plans include, for test protocols of the test experiment, indications of the test locations at which the seeds of the test sets of the test protocols are to be tested for the test experiment. In this manner, any number of seeds, test sets, test protocols, etc., may be allocated to any suitable number of test sites within a test network having any suitable number of test locations, regardless of the hubs to which the test sites are assigned (and thereby ensuring that parameters of the test protocol are appropriately and consistently applied). What's more, the tests sets (e.g., including a group of varieties, etc.) are directed into the suitable and representative locations for evaluation, thereby facilitating the collection of reliable data to support breeding advancement.

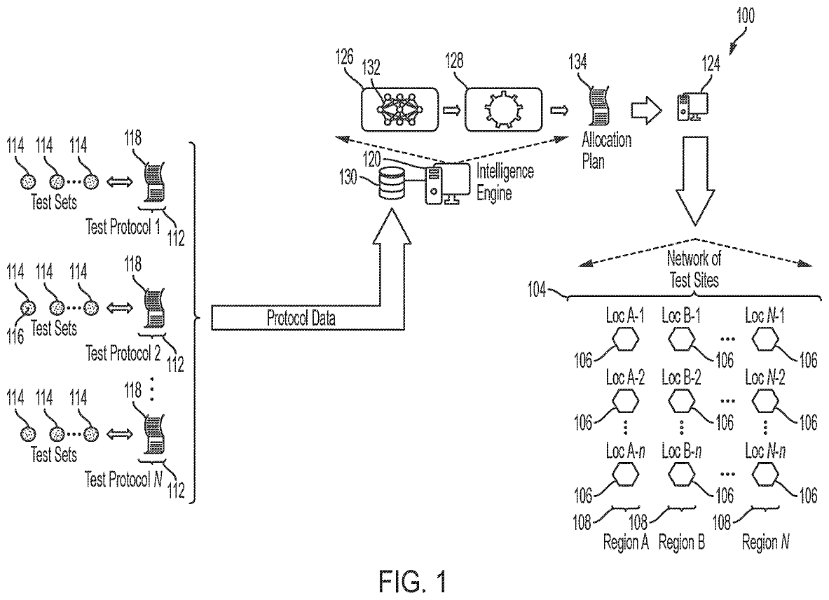

FIG. 1 illustrates an example system 100 in which one or more aspects of the present disclosure may be implemented. Although the system 100 is presented in one arrangement, other embodiments may include the parts of the system 100 (or additional parts) arranged or otherwise depending on, for example, the manner in which the breeding pipeline is arranged, number and/or arrangement of planting locations within a network of locations, types of seeds subject to planting and/or test experiments, etc.

In the example embodiment of FIG. 1, the system 100 generally includes a breeding pipeline (e.g., in which seeds are created and plants are grown from the seeds and tested, etc.) and a network 104 of locations 106 (e.g., test locations, other locations, etc.) at which desired tests may be performed in connection with seeds/plants associated with the breeding pipeline. The network 104, then, includes a plurality of regions 108, such that each of the regions 108 includes a plurality of the test locations 106. As an example, the system 100 may include four regions 108, where each of the regions 108 includes two-hundred test locations 106, resulting in a total of eight-hundred test locations 106. In other embodiments, the system 100 may include more or less than four regions 108, and the regions 108 may include different numbers of test locations 106. What's more, in various embodiments, the regions 108 may not all include the same number of test locations 106 (whereby some of the regions 108 may include more test locations 106 than other ones of the regions 108, etc.).

The test locations 106 each generally include a cultivation space, in which seeds 116 may be grown, matured, cultured, and/or cultivated, etc. (e.g., hybrid seeds, etc.). The cultivation spaces may each include any suitable area at any suitable location for cultivation of plants from the seeds 116, and may include, for example, pots, trays, grow rooms, greenhouses, plots, gardens, fields, combinations thereof, or the like, and may include indoor and/outdoor facilities. In addition, in certain embodiments, the plants grown from the seeds 116 may be cultured hydroponically at the test locations 106 in suitable aqueous media. In any case, the size and/or configuration of the cultivation spaces of the test locations 106 may be determined by those of ordinary skill in the art, and will often vary depending on the analyses to be performed, the seeds 116 to be analyzed, the regions 108 involved, etc.

At any given time, each of the test locations 106 is associated with one or more characteristics such as, for example, a geographic location (e.g., a latitude and/or a longitude, etc.), a region (e.g., an eastern, western, northern, or southern region of United States, etc.), a season (e.g., a summer, spring, fall, winter season, etc.), a size (e.g., a number of acres, a number of plots, etc.), a capacity (e.g., a maximum usable number of acres, etc.), a stage (e.g., early, mid, or late stage planting, etc.), plant type(s) (e.g., soy and/or corn hybrid plants, etc.), staffing (e.g., a number of testers, etc.), macro-environments (MACs) (e.g., MAC6.6, etc.), soil type(s) (e.g., s1727, etc.), product segment(s) (e.g., Delta, etc.), special rules (e.g., a rule that test protocols with maturity of −0.9 should not be allocated to locations in the United States, etc.), maturity (e.g., relative maturity of seeds/plants growing at the location, etc.), etc. In other embodiments, the test locations 106 may have or may be associated with more, fewer, and/or other characteristics, and the characteristics may vary from location to location.

The system 100 also includes a test experiment, as part of the breeding pipeline, for testing a plurality of the seeds 116. In connection therewith, the test experiment includes a plurality of test protocols 112. Each of the test protocols 112 has a different group of the test sets 114 assigned thereto, such that each of the test protocols 112 is specific to the group of test sets 114 assigned thereto. As an example (and without limitation), the test experiment may include one-thousand different test protocols 112, and each of the test protocols 112 may have ten test sets 114 assigned thereto. However, it should be appreciated that the same number of test sets need not be assigned to each test protocol 112 in all embodiments. It should also be appreciated that in one or more embodiments the system 100 may include multiple test experiments and that the disclosure herein is applicable to any suitable number to test experiments.

As described above, the test sets 114 each include a plurality of the seeds 116. Each of the test sets 114, then, represents the smallest unit of allocation to a test location 106, such that the seeds 116 are allocated to various test locations 106 on a set-by-set basis. That said, in one example, each of the test sets 114 may include between about sixty and about one-hundred and twenty different seeds 116 (or more or less), each of which is a different hybrid (e.g., a different hybrid of corn, soy, etc.). In the example test experiment, the test sets 114 assigned to each test protocol 112 include seeds that generally come from the same crop type but have different germplasms. In one or more other embodiments, the test sets 114 may be defined otherwise. Further, it should be appreciated, though, that the test sets 114 may each include the same or different numbers of seeds 116 and/or may each include different types of hybrids and/or types of seeds 116 in other examples.

The system 100 further includes a data structure 130 in, at, and/or associated with one or more of the breeding pipeline, the network 104, a region of test sites 106, the test sites 106 themselves, etc. In the example system 100, the data structure 130 is a cloud-based database, such that the protocol data may be downloaded from the database to a computer and then read into the intelligence engine 120 which is described in further detail below (e.g., using a python computer program, etc.). The data structure 130 is shown as a standalone part of the system 100. However, the data structure 130 may be incorporated in the intelligence engine 120, in whole or in part, or in other parts of the system 100 shown in FIG. 1, or otherwise. In various embodiments, the data structure 130 may be hosted, in whole or in part, in network-based memory (e.g., with Amazon Web Services, etc.) and/or in a dedicated computing device (e.g., stored locally, or remotely from the intelligence engine 120; etc.), whereby it is accessible to the intelligence engine 120 and/or users associated therewith via one or more networks. In various embodiments, the data structure 130 may be implemented as a PostgreSQL database, an Oracle database, or another type of database.

The data structure 130 includes protocol data for the test experiment, location data for the test locations 106, and historical allocation data for prior test experiments. The protocol data, location data, and historical allocation data may be fed into or retrieved by an intelligence engine 120 of the system 100, as described in greater detail below.

The protocol data is associated with each of the test protocols 112 of the test experiment. The protocol data includes test requirements and test set characteristics for the seeds 116 in the test sets 114 subject to the given test protocol 112 (and corresponding values, etc.). That said, the seeds 116 are generally assigned to an appropriate one of the test locations 106 based on the characteristics of the seeds 116 and/or the test protocol 112, and are then subjected to the requirements of the corresponding protocol data in execution of the test experiment at the assigned test location.

Table 1 includes example test requirements (e.g., parameters, etc.) that may be included in protocol data for a test protocol 112 (in association with their respective description (or variables), whereby appropriate values may then be assigned to each of the requirements for the seeds at the given test site (and stored as desired)). It should be appreciated, though, that the requirements included in Table 1 are example only, and that the test protocol may include different, additional, etc. requirements in other examples. In connection therewith, it should also be appreciated that the protocol data (including its requirements) may not be consistent across all of the test protocols 112. That said, in the example test experiment, the test requirements are generally the same for each test set 114 assigned to a given test protocol 112.

TABLE 1

| Test Requirement | Description |
|---|---|
| Region | Region (e.g., United States (US), etc.) of test location(s) 106 in |
| | which test sets 114 assigned to the test protocol 112 are to be |
| | tested. |
| Planting Year | Year during which test sets 114 assigned to the test protocol |
| | 112 are to be planted. |
| Environment | Desired Macro Environments (MACs) in which the test sets are |
| | to be tested (e.g., MAC9, MAC1.2, etc.). |
| Plots per site (or location) | Spacing of seed/plant rows required at each test location 106. |
| Plots per length | Plots, per length of test location, required at each test location |
| | 106. |
| Row spacing | Spacing of seed/plant rows required at each test location 106. |
| Area per site | Acres required at each test location 106. |
| Relative Maturity (RM) | Maximum RM allowed for other seeds/plants at each test |
| Maximum | location 106. |
| Relative Maturity (RM) | Minimum RM allowed for other seeds/plants at each test |
| Minimum | location 106. |

Table 2 includes example test set characteristics that may be included in the protocol data for a test protocol 112 (in association with their respective description (or variables), whereby appropriate values may then be assigned to each of the characteristics for the seeds at the given test site). It should be appreciated, though, that the characteristics included in Table 2 are example only, and that the test protocol 112 may include different, additional, etc. characteristics in other examples. In connection therewith, it should also be appreciated that the protocol data (and/or the characteristics thereof) may not be consistent across all of the test protocols 112 in the system 100. That said, in the example test experiment, the test set characteristics are generally the same for each test set 114 assigned to or associated with a given test protocol 112 of the test experiment.

TABLE 2

| Test Set Characteristic | Description |
|---|---|
| Protocol ID | Identifier for the corresponding test protocol 112 (e.g., a unique |
| | identifier, a substantially unique identifier, an identifier that is |
| | unique or substantially unique within the test experiment, etc.). |
| Protocol Name | Name for the corresponding test protocol 112. |
| Test Set Names | Names for the test sets 114 assigned to the test protocol 112 (e.g., |
| | names that are different from and/or independent of the Protocol ID, |
| | etc.). |
| Crops | Crop types (e.g., “soybeans”, “corn,” etc.) for seeds 116 assigned to |
| | the test sets 114 of the test protocol 112. |
| Organization | An organization, entity, or group (e.g., Breeding,” “Breeding, |
| | TechDev,” etc.) associated with the test protocol 112 (e.g., |
| | responsible for overseeing testing of seeds 116 of the test sets 114 |
| | assigned to the test protocol 112, etc.). |
| Crop Material Stage | Stage of the development of seeds 104 assigned to or associated with |
| | the test protocol 112 (e.g., “Screening 1, “Screening 2,” “Pre- |
| | Commercial 1,” “Pre-commercial 2,” “Pre-commercial 3,” “Pre- |
| | Commercial 4,” etc.). |
| Trial Type | Type of trial being conducted pursuant to the test experiment for test |
| | sets assigned to the test protocol (e.g., “Field Trial, etc.”). |
| Trial Intent | Intent of the trial being conducted pursuant to the test experiment for |
| | test sets 114 assigned to the test protocol 112 (e.g., “Standard Yield |
| | BAY/Gxe,” etc.). |
| Compliance Type | Compliance type for seeds 104 assigned to the test protocol 112 |
| | (e.g., “Approved Trait,” “Non-Trained,” “Stewarded Seed,” etc.). |
| Relative Maturity (RM) | RM of seeds 116 assigned to the test protocol 112. |
| Trait | Trait(s) of seeds 116 assigned to the test protocol 112 (e.g., shared |
| | traits/characteristics by which the seeds 116 of the test sets 114 are |
| | grouped into the test sets 114 and by which the test sets 114 are |
| | assigned to or associated with (e.g., grouped into) the test protocol |
| | 112, such as RR2Y, RR2X, etc.). |

Table 3 includes example location data for a test location 106 (in association with respective descriptions (or variables), whereby appropriate values may then be assigned to each of the variables for the seeds 116 at the given test location 106). It should be appreciated that the location data included in Table 3 is example only, and that test locations 106 may include other data in other examples. It should also be appreciated that one or more of the test locations 106 in the system 100 may include different location data than other ones of the test locations 106.

TABLE 3

| Location Data | Description |
|---|---|
| Location ID | Identifier for the test location 1056 (e.g., a unique identifier, a |
| | substantially unique identifier, an identifier that is unique or |
| | substantially unique within the network of test locations, etc.). |
| Crop Material Stage | All stages of development of plants/seeds that are permitted at the |
| | test location 106 (e.g., “Screening 1, “Screening 2,” “Pre- |
| | Commercial 1,” “Pre-commercial 2,” “Pre-commercial 3,” “Pre- |
| | Commercial 4,” etc.). |
| availableDate | Date on or after which the test location 106 is available to accept |
| | new test sets (e.g., “≥YYYY-MM-DD”, etc.). |
| Year | Year in which the test location 106 is available to accept new test |
| | sets. |
| Status | Whether the test location 106 is available to accept new test sets 114 |
| | (e.g., “AVAILABLE,” etc.) |
| Macro-environment | Macro-environment(s) where the test location 106 is geographically |
| (MAC) | located (e.g., MAC9, MAC1.2, etc.). |
| Soil Type | Soil type at the test location 106 (e.g., s1897, etc.). |
| Relative Maturity (RM) | Relative maturity of plants/seeds planted or growing at the test |
| | location 106. |
| Capacity | Capacity of the test location 106 (e.g., 40 acres etc.). |
| Home Location | An indication of whether the test location 106 is a home location of |
| | the corresponding hub for the test location 106 (e.g., geographically |
| | close to the individuals associated with and equipment of the hub, |
| | etc.). |
| gps_Point | Global positioning system (GPS) coordinates of the test location 106 |
| | (e.g., latitude and longitude, etc.). |
| cropName | Name(s) of crop(s) currently planted or tested at the test location |
| | 106 (e.g., Corn, Soybeans, etc.). |
| cropTrialType | Type(s) of trial(s) currently being conducted at the test location |
| | (e.g., “Field Trial,” etc.). |
| ComplianceType | Compliance type(s) for plants/seeds planted or growing at the test |
| | location 106 (e.g., “Approved Trait,” “Non-Trained,” “Stewarded |
| | Seed,” etc.). |

The historical allocation data, then, generally includes, for each of a plurality of prior test experiments, one or more historical protocol requirements (e.g., consistent with one or more test protocol requirements listed above in Table 1, etc.), one or more historical test set characteristics (e.g., consistent with one or test set protocol characteristics listed above in Table 2, etc.), and/or prior test location data for each test location 106 (e.g., consistent with one or more items of location data listed above in Table 3, etc.). Example historical allocation data is illustrated in Table 4.

TABLE 4

| Historical Data | Description (Per Test Experiment) |
|---|---|
| Location ID | Identifier(s) for the test location(s) 106 for the prior test experiment |
| | (e.g., a unique identifier, a substantially unique identifier, an |
| | identifier that is unique or substantially unique within the network |
| | of test locations, etc.). |
| Crop Material Stage | Stage of the development of prior seeds assigned to each prior test |
| | protocol 112 (e.g., “Screening 1, “Screening 2,” “Pre-Commercial |
| | 1,” “Pre-commercial 2,” “Pre-commercial 3,” “Pre-Commercial 4,” |
| | etc.). |
| Relative Maturity (RM) | RM of seeds assigned to each prior test protocol 112 of the prior |
| | test experiment (for each prior test set). |
| Season | Season during which plants/seeds were grown at the test location(s) |
| | 106 (for each prior test set assigned to or associated with each prior |
| | test protocol of the prior test experiment), such as Winter, Summer, |
| | Spring, Fall, etc.). |
| Home Location | An indication of whether the test location(s) 106 for the prior test |
| | experiment is/was a home location of the corresponding hub for the |
| | test location(s) (e.g., geographically close to the individuals |
| | associated with and equipment of the hub, etc.). |
| Latitude | Latitude of the test location(s) 106 for the prior test experiment. |
| Longitude | Longitude of the test location(s) 106 for the prior test experiment. |
| Maturity Difference | Difference (e.g., the absolute value of the difference, etc.) between |
| | the RM of seeds 116 in the prior test set 114 and the RM of |
| | plants/seeds at the test location(s) 106 to which the prior test set(s) |
| | 114 was/were allocated. |

It should be appreciated that the prior test experiments (associated with the historical allocation data) may differ from the test experiments described above (broadly, the current test experiments) in that prior test sets may be assigned to prior test protocols of the prior test experiment, such that the prior test protocols have already been allocated to test locations 106 (e.g., the prior seeds of each prior test set of the prior test protocols have already been planted, grown, matured, and/or cultured, etc. at locations 106 as part of the prior test experiments, etc.).

With continued reference to FIG. 1, the breeding pipeline is arranged such that seeds 116 are bread (broadly, created) therein, generated and then allocated to the test locations 106, where the seeds are grown and tested. In connection therewith, the example system 100 includes the intelligence engine 120. As described in greater detail below, the intelligence engine 120 is generally configured to, based, at least in in part, on the test protocol requirements, location data, and historical protocol data, automatically allocate the test protocols 112 among the test locations 106 without manual assignment by a user.

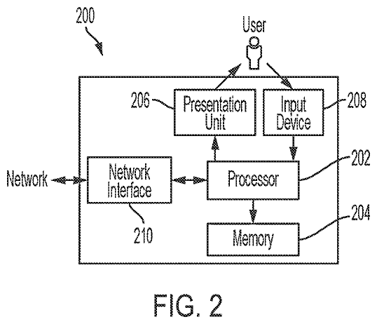

FIG. 2 illustrates an example computing device 200 that can be used in the system 100. The computing device 200 may include, for example, one or more servers, workstations, personal computers, laptops, tablets, smartphones, virtual devices, etc. In addition, the computing device 200 may include a single computing device, or it may include multiple computing devices located in close proximity or distributed over a geographic region, so long as the computing devices are specifically configured to operate as described herein. In the example embodiment of FIG. 1, the intelligence engine 120 may include (or may be implemented in) one or more computing devices consistent with computing device 200. Also, in the example embodiment, the system 100 includes the data structure 130 and a capacity reservation system 124 (described in greater detail below), each of which may be understood to be consistent with the computing device 200 and/or implemented in a computing device consistent with computing device 200 (or implemented in a part thereof, such as, for example, memory 204, etc.). However, the system 100 should not be considered to be limited to the computing device 200, as described below, as different computing devices and/or arrangements of computing devices may be used. In addition, different components and/or arrangements of components may be used in other computing devices.

As shown in FIG. 2, the example computing device 200 includes a processor 202 and a memory 204 coupled to (and in communication with) the processor 202. The processor 202 may include one or more processing units (e.g., in a multi-core configuration, etc.). For example, the processor 202 may include, without limitation, a central processing unit (CPU), a microcontroller, a reduced instruction set computer (RISC) processor, a graphics processing unit (GPU), an application specific integrated circuit (ASIC), a programmable logic device (PLD), a gate array, and/or any other circuit or processor capable of the functions described herein.

The memory 204, as described herein, is one or more devices that permit data, instructions, etc., to be stored therein and retrieved therefrom. In connection therewith, the memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable media for storing such data, instructions, etc. In particular herein, the memory 204 is configured to store data including, without limitation, protocol data (e.g., test protocol requirements, test set characteristics, etc.), test location data, models (e.g., a first stage machine learning prediction model (MLPM) and second stage optimization model (OM), etc.), neural networks, training data for the models and/or neural networks (e.g., historical allocation data, etc.), input and output data for the models (e.g. for the models and/or neural networks (e.g., allocation prediction scores, allocation probability matrices, allocation plans, etc.), and/or other types of data (and/or data structures) suitable for use as described herein. Furthermore, in various embodiments, computer-executable instructions may be stored in the memory 204 for execution by the processor 202 to cause the processor 202 to perform one or more of the operations described herein (e.g., in method 500, etc.) in connection with the various different parts of the system 100, such that the memory 204 is a physical, tangible, and non-transitory computer readable storage media. Such instructions often improve the efficiencies and/or performance of the processor 202 that is performing one or more of the various operations herein, whereby in connection with performing the operations the computing device 200 may be transformed into a special purpose computing device. It should be appreciated that the memory 204 may include a variety of different memories, each implemented in connection with one or more of the functions or processes described herein.

In the example embodiment, the computing device 200 also includes a presentation unit 206 that is coupled to (and is in communication with) the processor 202 (however, it should be appreciated that the computing device 200 could include output devices other than the presentation unit 206, etc.). The presentation unit 206 may output information (e.g., interactive interfaces, etc.), visually or otherwise, to a user of the computing device 200, such as a breeder, tester, or other person associated with a test experiment, the intelligence engine 120, and/or the allocation of test protocols 112 to test locations 106, etc. It should be further appreciated that various interfaces (e.g., as defined by network-based applications, websites, etc.) may be displayed at computing device 200, and in particular at presentation unit 206, to display certain information to the user. The presentation unit 206 may include, without limitation, a liquid crystal display (LCD), a light-emitting diode (LED) display, an organic LED (OLED) display, an “electronic ink” display, speakers, etc. In some embodiments, presentation unit 206 may include multiple devices. Additionally or alternatively, the presentation unit 206 may include printing capability, enabling the computing device 200 to print text, images, and the like on paper and/or other similar media.

In addition, the computing device 200 includes an input device 208 that receives inputs from the user (i.e., user inputs). The input device 208 may include a single input device or multiple input devices. The input device 208 is coupled to (and is in communication with) the processor 202 and may include, for example, one or more of a keyboard, a pointing device, a mouse, a stylus, a touch sensitive panel (e.g., a touch pad or a touch screen, etc.), or other suitable user input devices. It should be appreciated that in at least one embodiment an input device 208 may be integrated and/or included with an output device 206 (e.g., a touchscreen display, etc.).

Further, the illustrated computing device 200 also includes a network interface 210 coupled to (and in communication with) the processor 202 and the memory 204. The network interface 210 may include, without limitation, a wired network adapter, a wireless network adapter, a mobile network adapter, or other device capable of communicating to one or more different networks (e.g., one or more of a local area network (LAN), a wide area network (WAN) (e.g., the Internet, etc.), a mobile network, a virtual network, and/or another suitable public and/or private network capable of supporting wired and/or wireless communication among two or more of the parts illustrated in FIG. 1, etc.), including with other computing device used as described herein.

Referring again to FIG. 1, the intelligence engine 120 and the capacity reservation system 124 of the system 100 are each specifically configured by computer executable instructions to perform one or more of the operations described herein. In the illustrated embodiment, the intelligence engine 120 and the capacity reservation system 124 are both shown as standalone parts of the system 100. However, in various other embodiments, it should be appreciated that the intelligence engine 120 and/or the capacity reservation system 124 may be associated with, or incorporated with, other parts of the system 100, for example, the breeding pipeline, etc. In various embodiments, the intelligence engine 120 and/or the capacity reservation system 124 may be embodied in at least one computing device and may be accessible as a network service (e.g., a cloud-based web service such as Amazon Web Services, etc.), via, for example, an application programming interface (API), or otherwise, etc.

The intelligence engine 120 includes (e.g., in a memory 204 thereof, etc.) a first stage machine learning prediction model (MLPM) 126 and a second stage optimization model (OM) 128. As described in greater detail below, the intelligence engine 120 is configured train the first and second stages models 126 and 128 and then to, based on the test protocols 112, execute the first stage MLPM 126 to automatically generate an output that includes allocation preference scores. The allocation preference scores indicate probabilities that the test locations 106 will satisfy the test protocols 112. In addition, the intelligence engine 120 is configured to then, based on the output of the first stage MLPM 126 (and, in particular, the allocation preference scores), execute the second stage OM 128 to automatically generate an output that includes an allocation plan for the test sets 114 assigned to the test protocols 112. That said, the stages of the models 126 and 128 are not to be confused with the stage (e.g., crop material stage, etc.) of the test locations 106 or seeds 104 (e.g., as included in the test location data and/or protocol data, etc.).

The example first stage MLPM 126 includes a recurrent neural network 132. The first stage MLPM 126 may constructed from and/or implemented with any of a variety of machine learning algorithms, libraries, models and/or software known in the art such as, for example, the PyTorch open source machine learning library, etc. to facilitate such training. Further, the example recurrent neural network 132 is based on a long short-term memory (LSTM) architecture which is advantageously capable of processing entire sequences of data. For example, the LSTM architecture may be an artificial recurrent neural network (RNN) for deep learning, which uses feedback connections and is capable of processing single data points or entire sequences of data. Each LSTM unit may include a cell, an input gate, an output gate, a forget gate, etc. In one or more other embodiments, the recurrent neural network 132 may be based on a different architecture.

The intelligence engine 120 is configured to retrieve or receive the historical allocation data for the plurality of prior test experiments from the data structure 130 and train the recurrent neural network 132 using the historical allocation data. It should be appreciated that, in some embodiments, the historical allocation data may be based (at least in part) on manual allocations (e.g., performed by personnel associated with the hubs assigned to the test locations 106, etc.). This may be the case, for example, where the intelligence engine 120 has not previously executed the models 126 and 128 to generate an allocation plan for test sets 114 assigned to test protocols 112 of a test experiment. However, the intelligence engine 120 may be configured, after generating an allocation plan for a current test experiment, to update the historical allocation data to include the protocol data for each test protocol 112 of the current test experiment, thereby automatically updating the allocation plan generated as an output, the test location data for the test locations 106 in which the test experiment is (or is to be) executed, and the allocation plan generated as the output of the second stage OM 128. The intelligence engine 120 may be configured to then store the updated historical allocation data in the data structure 130.

For example, the intelligence engine 120 may divide the historical allocation data (which may include manually assigned previous allocations and/or prior allocation plans 134 generated by the models 126), into training and testing data sets. The recurrent neural network 132 and/or models 126 and 128 may be trained using any suitable machine learning, etc., techniques, such as supplying the training data set to the recurrent neural network 132 and/or models 126 and 128 with the test protocol requirements and the test set characteristics of the historical data used as inputs (e.g., the protocol data listed above in Tables 1 and 2, etc.), then comparing the output of the recurrent neural network 132 and/or models 126 and 128 with output allocation plans from the historical allocation data. The testing data sets may be used to test the accuracy of the trained recurrent neural network 132 and/or models 126 and 128, and until the network 132 and/or models 126 and 128 reach a desired accuracy threshold. Parameters of the network 132 and/or models 126 and 128 may be adjusted during training according to any suitable machine learning techniques, etc.

With the recurrent neural network 132 of the first stage MLPM 126 being trained, the intelligence engine 120 is configured to receive and/or retrieve the protocol data for each test protocol 112 and, in particular, the test protocol requirements and the test set characteristics for each test protocol 112 (e.g., the protocol data listed above in Tables 1 and 2, etc.). The intelligence engine 120 is configured to receive and/or retrieve the protocol data via an API (e.g., an Apache Velocity API, etc.), for example, associated with the network 104, etc. or otherwise. Alternatively, in at least one embodiment, the data for the test protocols 112 may be created in or by the intelligence engine 120.

The intelligence engine 120 is additionally configured to receive and/or retrieve the test location data for each of the test locations 106 from the data structure 130 (e.g., from the test locations 106, from the network 104, etc.). The test location data generally includes, for each test location 106 in the network 104, the location data described above (e.g., one or more of the items listed in Table 3, one or more additional items or other items, etc.). The intelligence engine 120 is configured to receive and/or retrieve the test location data, again, via an API (e.g., an Elasticsearch API, etc.) or otherwise. Alternatively, again, the test location data may be created in or by the intelligence engine 120.

Next in the system 100, the intelligence engine 120 is configured to execute the first stage MLPM 126 based on the protocol data and the test location data to generate a first stage output. In general, the first stage output includes, for each of the plurality of test locations 106 (e.g., as defined in the test location data, etc.), an allocation preference score (broadly, an allocation prediction score) for each test protocol 112 of the current test experiment. Each allocation prediction score represents a probability, based on the historical allocation data and the trained first stage MLPM 126, that the test protocol 112 should be allocated or advanced to the corresponding test location 106. It should be appreciated that each allocation prediction score may additionally or alternatively be viewed as representing a preference, based on the historical allocation data and the trained first stage MLPM 126, that the test protocol 112 will be allocated or advanced to the corresponding test location 106. Further, the score may additionally or alternatively be viewed as representing a probability that the corresponding test location 106 satisfies the protocol data and, in particular, that the test location 106 (as represented in the test location data) meets or satisfies the test requirements for the protocol data for the test protocol 112 and is compatible with the characteristics of the seeds 116 of the test sets 114 assigned to the test protocol 112. In either case, the score, prediction, and/or probability is based on a combination of the historical allocation data, the test location data, and the protocol data for the corresponding test protocol 112.

The first stage output may also include (or may be arranged as) a probability matrix. In doing so, for example, the probability matrix may represent multiple different allocation prediction scores for a given test protocol 112. In particular, the probability matrix may include an allocation prediction score for each of the plurality of test protocols 112 of the current test experiment. In this way, in this example, the probability matrix is generally assigned to or associated with the given test protocol. In one or more other examples, the first stage output may be structured and/or arranged in one or more other manners and/or may represent the probabilities in one or more other fashions.

Table 5 illustrates multiple example probability matrices that may be generated by the intelligence engine 120 in executing the first stage MLPM 126 for each of multiple test protocols 112 (Protocols 1 through N). The example matrices each include allocation prediction scores for each of multiple test locations 106, where each score is expressed as a number in a range of zero to one. In connection therewith, an allocation prediction score of zero indicates no probability that the given test protocol 112 (e.g., Protocol 1, Protocol 2, etc.) would have been assigned to the corresponding test location 106 (e.g., Loc A-1, Loc A-2, etc.). An allocation prediction score of 0.5 indicates a 50% probability that the given test protocol 112 would have been assigned to the corresponding test location 106. And, an allocation prediction score of 1.0 indicates a 100% probability that the given test protocol 112 would have been assigned to the corresponding test location 106. As described above, the allocation prediction scores may be generated from the recurrent neural network 132 of the first stage MLPM 126, etc.

TABLE 5

| | Loc A-1 | Loc A-2 | . . . | Loc A-n |
|---|---|---|---|---|
| Protocol 1 | 0.5 | 0.1 | . . . | 0.8 |
| Protocol 2 | 0.4 | 0.2 | . . . | 0.7 |
| . . . | . . . | . . . | . . . | . . . |
| Protocol N | 0.9 | 0.0 | . . . | 0.5 |

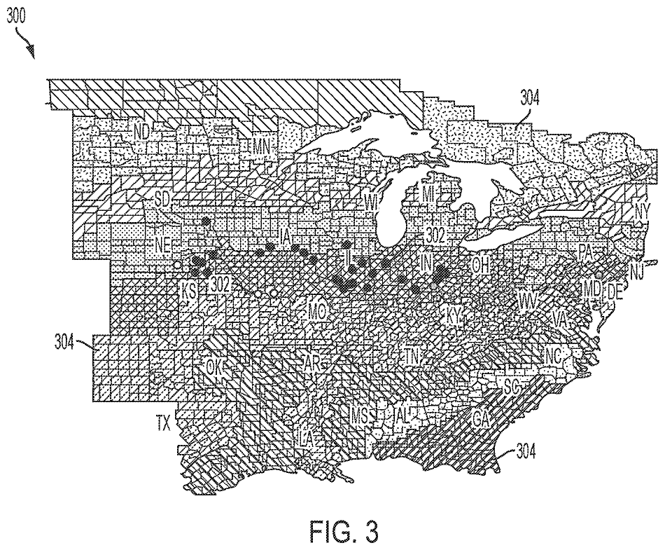

FIG. 3 illustrates an example map 300. The map 300 illustrates numerous regions 304 (e.g., multiple regions encompassing each state (e.g., MO, AR, TN, etc.)) that are each shaded according to the relative maturity of plants/seeds that are planted/growing at one or more test locations 106 within the region 304. The darkest shaded regions 304 are those where plants/seeds with a higher relative maturity (RM) (e.g., RM 7, etc.) are planted/growing at one or more test locations 106 within the region. The lighter the shading of a regions 304, the lower the relative maturity (RM) of plants/seeds that are planted/growing at one or more test locations 106 within the region 304.

The map 300 then also provides a visualization based on a first stage output, by the intelligence engine 120, of allocation prediction scores (following execution of the first stage MLPM 126). More specifically, the map 300 includes a plurality probability indicators 302 each associated with a test location 106 within a region 304. The probability indicators 302 represent a probability (e.g., an allocation prediction score, etc.) that a test protocol 112 of a test experiment should be assigned to the test location 106 corresponding to the probably indicator 302, where the test location 106 also corresponds to the indicated relative maturity (RM). In doing so, in this embodiment, such probability is based on a shading of the probability indicators 302. The lighter in shade the probability indicator 302, the lower the probability that the test set 114 should be assigned or allocated to the corresponding test location 106. The darker the probability indicator 302, the higher the probability that the test set 114 should be assigned or allocated to the corresponding test location 106.

Referring again to FIG. 1, the intelligence engine 120 is configured, after executing the trained first stage MLPM 126 (to generate the first stage output), to store the first stage output and, in particular, the probability matrix representative thereof, in the data structure 130, or otherwise, for subsequent use as described below. In one or more other embodiments, though, the first stage output need not necessarily be stored in the data structure 130.

The intelligence engine 120 is configured then to execute the second stage OM 128. The second stage OM 128 generally includes a plurality of objective functions and a plurality of constraints. The objective functions include a plurality of multi-objective mixed-integer programming problems. As such, in executing the second stage OM 128, the intelligence engine 120 is configured to execute the plurality of objective functions, subject to the plurality of constraints, based on a plurality of indices and sets, a plurality of function parameters, and a plurality of decision variables. The second stage OM 128 may be constructed from and/or implemented with any of a variety of optimization models, libraries, and/or software known in the art such as, for example, the IBM ILOG CPLEX Optimization Studio, etc.

Table 6 includes multiple example indices and sets that may be utilized by the intelligence engine 120 in connection with execution of the second stage OM 128, with respect to a given test experiment.

TABLE 6

| Indices or Sets | Description |
|---|---|
| i | Test protocol index, where i ∈ I |
| j | Test location index, where j ∈ J |
| s | Stage index (e.g., crop material stage index, etc.), where s ∈ S, and where |
| | the stage index may be represent an index of the stages of development of |
| | seeds 116 assigned to the test protocols 112 and/or an index of all stage of |
| | development of the plants/seeds that are permitted at the test locations 106 |
| I | Set of test protocols 112 included in test experiment |
| Iₛ | Set of test protocols 112 for stage s |
| I_{sga} | Set of test protocols for stage s, RM group g, and trait a |
| J | Set of test locations 106 within network 104 |
| J^{h} | Set of test locations 106 that belong to/are assigned to hub h |
| J^{hm} | Set of home locations |
| J^{e} | Set of test protocols 112 in an east region of the network 104 |
| J^{w} | Set of test protocols 112 in a west region of the network 104 |
| J^{m} | Set of test locations that have MAC m |
| J^{t} | Set of test locations within the network that have soil type t |
| J^{g} | Set of test locations within the network 104 that have product segment g |
| S | Set of stages (e.g., stages s (e.g., crop material stages, etc.), etc.), where S is |
| | the set of all possible stages of development of seeds 116 assigned to the |
| | test protocols 112 and/or all stage of development of the plants/seeds that |
| | are permitted at the test locations 106 within the network 104 |
| S_{sc} | Set of screen stages |
| S_{pc} | Set of pre-commercial stages |
| H | Set of hubs |
| R | Set of maturity group(s) |
| T | Set of traits |
| MC | Set of macro-environments (MACs) |
| PS | Set of product segments |
| SL | Set of soil types |
| SP | Set of special rules |

Table 7 includes multiple example function parameters that may be utilized by the intelligence engine 120 in connection with execution of the second stage OM 128 (together with the example indices and sets of Table 6), with respect to a given test experiment.

TABLE 7

| Function Parameters | Description |
|---|---|
| [symbol] | Logic function return, where [symbol] = 1 if the statement is true; otherwise, |
| | [symbol] = 0 |
| λₛ^{ξ} | Weight for objective o at stage s, where ξ ∈ {set of objectives}, |
| | and where the set of objectives refer to different objectives (e.g., the |
| | objective functions and/or descriptions of Table 9) |
| RMᵢ^{p} | RM for test protocol 112 i |
| RMⱼ^{l} | RM for test location 106 j |
| MKTⱼ | Normalized sales data for test location 106 j |
| PSD_{g} | Ideal percentage of test locations 106 at product segment g |
| PLTᵢ | Plots for test protocol 112 i |
| ACᵢ | Acres required for test protocol 112 i |
| MUAⱼ | Maximum usable acres of test location 106 j |
| Nᵢ | Number of test locations 106 required by test protocol 112 i |
| Cₕ^{U} | Capacity upper bound of hub h |
| Cₕ^{L} | Capacity lower bound of hub h |
| M | Constant number |
| Pᵢⱼ | Predicted allocation likelihood (e.g., as represented by the allocation |
| | prediction score generated as an output of the first stage MLPM |
| | 126,, etc.) for protocol 112 i and test location 106 j) |

Table 8 includes multiple example decision variables that may be utilized by the intelligence engine 120 in connection with execution of the second stage OM 128 (together with the example indices and sets of Table 6 and the example function parameters of Table 7), with respect to a given test experiment.

TABLE 8

| Decision | |
|---|---|
| Variables | Description |
| xᵢⱼ | Indication of whether test protocol 112 i will be assigned |
| | to test location 106 j, as generated as an output of the |
| | second stage MLPM 128, where the test protocol 112 i |
| | (containing multiple test sets 114 that are generally the |
| | same) is generally assigned to multiple locations j of |
| | the set of test locations J within the network 104. |
| Zᵢ^{r} | Penalty for breaking special rule r at protocol i |

Table 9 includes multiple objective functions and, in particular, multi-objective mixed integer linear programming problems, which may be utilized by the intelligence engine 120 in connection with execution of the second stage OM 128, with respect to a given test experiment. In connection therewith, application of the object functions in executing the second stage OM 128 is generally based on one or more of the example indices and/or sets of Table 6, the example function parameters of Table 7, and/or the example decision variables of Table 8.

TABLE 9

| | Objective Function | Description |
|---|---|---|
| (a) | $min {\sum }_{s ∈ S} {λ}_{s}^{a} {\sum }_{i ∈ {I}_{s}} {\sum }_{i ∈ J} {x}_{ij} \cdot \vert {RM}_{i}^{p} - {RM}_{j}^{l} \vert$ | RM of test protocol 112 should be close to core RM of test location 106 |
| (b) | $- {\sum }_{s ∈ S} {λ}_{s}^{b} {\sum }_{i ∈ {I}_{s}} {\sum }_{i ∈ J} {x}_{ij} {MKT}_{j}$ | Direct test protocol 112 to test locations 106 that represent large market |
| (c) | $+ {\sum }_{s ∈ {S}_{c}} {λ}_{s}^{c} {\sum }_{j ∈ J} ( {\sum }_{i ∈ {I}_{s}} {x}_{ij} \geq 1 )$ | Minimize number of screening locations |
| (d) | $- {\sum }_{s ∈ {S}_{pc}} {λ}_{s}^{d} {\sum }_{j ∈ J} ( {\sum }_{i ∈ {I}_{s}} {x}_{ij} \geq 1 )$ | Maximize number of pre-commercial locations |
| (e) | $+ {\sum }_{s ∈ S} {λ}_{s}^{e} {\sum }_{i ∈ {I}_{s}} \vert {\sum }_{j ∈ {J}^{e}} {x}_{ij} - {\sum }_{j ∈ {J}^{w}} {x}_{ij} \vert$ | Balance number of east and west assignments |
| (f) | $+ {λ}^{f} {\sum }_{i ∈ I} {\sum }_{m ∈ MC} max \{ {\sum }_{j ∈ {J}^{m}} {x}_{ij} - 1 , 0 \}$ | Minimize duplication reps of one test protocol 112 into same macro-environment (MAC), such that repetitive allocation of a test |
| | | protocol 112 (e.g., allocation of multiple test |
| | | sets 114 of the test protocol 112, etc.) to the |
| | | same test location 106 (e.g., a test location |
| | | that that permits “double planting,” etc.) is |
| | | minimized |
| (g) | $+ {λ}^{g} {\sum }_{i ∈ I} {\sum }_{t ∈ SL} max \{ {\sum }_{j ∈ {J}^{t}} {x}_{ij} - 1 , 0 \}$ | Minimize duplication reps of one test protocol 112 into same soil type |
| (h) | $+ {λ}^{h} {\sum }_{i ∈ {I}_{s}} {\sum }_{j ∈ {J}^{hm}} {x}_{ij}$ | Home locations are preferred for screening protocol |
| (i) | $+ {λ}_{r}^{i} {\sum }_{r ∈ SP} {\sum }_{i ∈ I} {Z}_{i}^{r}$ | Minimize special rules' penalty |
| (j) | $- {\sum }_{s ∈ S} {λ}_{s}^{j} {\sum }_{i ∈ {I}_{s}} {\sum }_{j ∈ {J}_{s}} {x}_{ij} \cdot {P}_{ij}$ | Allocations with higher predicted allocation likelihood (e.g., as reflected in the output of the first stage MLPM 126, etc.) are preferred |
| (k) | $+ {\sum }_{s ∈ S} {λ}_{g}^{j} {\sum }_{i ∈ {I}_{s}} {\sum }_{g ∈ {P}_{s}} \vert {PSD}_{g} - {\sum }_{j ∈ {J}^{g}} {x}_{ij} \div {N}_{i} \vert$ | Allocation distribution should follow ideal distribution over each product segment |

And, Table 10 includes example constraints that may be utilized by the intelligence engine 120 in connection with execution of the second stage OM 128 (whereby the intelligence engine 120 may be configured to execute the example objective functions of Table 9 subject to the example constraints included in Table 10), with respect to a given test experiment. In connection therewith, the example constraints are generally based on one or more of the example indices and/or sets of Table 6, the example function parameters of Table 7, and/or the example decision variables of Table 8.

TABLE 10

| | Constraint | Description |
|---|---|---|
| (1) | ${\sum }_{j ∈ J} {x}_{ij} = {N}_{i} , ∀ i ∈ I$ | Test protocol 112 should be placed to needed number of test locations |
| (2) | xᵢⱼ = 0, ∀i ∈ {i · complaince ≠ | Test protocol 112's stage and |
| | j · compliance, i · stage ∉ j · stage} | compliance type need to match stage |
| | | and compliance type of test location 106 |
| (3) | ${C}_{h}^{L} \leq {\sum }_{i ∈ I} {\sum }_{j ∈ {J}^{h}} {x}_{ij} \cdot {PLT}_{i} \leq {C}_{h}^{U} , ∀ h ∈ H$ | Total plots for each hub should be between the lower bound and the upper bound of the hub's capacity |
| (4) | ${\sum }_{i ∈ I} {x}_{ij} \cdot {AC}_{i} \leq {MUA}_{j} , ∀ j ∈ J$ | Total acres used for each test location 106 should be no more than of the test location 106's maximum |
| | | usable area |
| (5) | $\begin{matrix}∀ {a}_{1} ∈ T , ∀ {a}_{2} ∈ T , ∀ s ∈ S , ∀ j ∈ J , ∀ g ∈ R \\ \{ \begin{matrix}{x}_{{i}_{1} , j \leq M \cdot {x}_{{i}_{2} , j} if {N}_{{i}_{1} \leq {N}_{{i}_{2}}}} \\ {x}_{{i}_{1} , j \geq M \cdot {x}_{{i}_{2} , j} if {N}_{{i}_{1} \geq {N}_{{i}_{2}}}}\end{matrix} ∀ {i}_{1} ∈ {I}_{s , g , a ,} ∀ {i}_{2} ∈ {I}_{s , g , {a}_{2}}\end{matrix}$ | Test protocols 112 with the same stage, same maturity, and different traits should be allocated to the same test locations 106 |
| (6) | $\begin{matrix}∀ s ∈ S , ∀ a ∈ T , ∀ j ∈ J , ∀ g ∈ R \\ \{ \begin{matrix}{x}_{{i}_{1} , j \leq M \cdot {x}_{{i}_{2} , j} if {N}_{{i}_{1} \leq {N}_{{i}_{2}}}} \\ {x}_{{i}_{1} , j \geq M \cdot {x}_{{i}_{2} , j} if {N}_{{i}_{1} \geq {N}_{{i}_{2}}}}\end{matrix} ∀ {i}_{1} ∈ {I}_{s , g , a ,} ∀ {i}_{2} ∈ {I}_{s + 1 , g , a}\end{matrix}$ | Test protocols 112 should be co- located with later stage test protocols 112 if they are from the same RM group and the same trait |
| (7) | xᵢⱼ ≤ zᵢ^{r}, ∀i ∈ Iₛ, ∀j ∈ | One or more subsets of test |
| | {(e. g., excluded location)} | protocols 112 should follow one or |
| | | more rules (e.g., screening protocols |
| | | should not go to Ontario test locations); |
| | | the special rules may be inconstant |

That said, before executing the second stage OM 128, the intelligence engine 120 is configured to retrieve the first stage output generated by the first stage MLPM 126 and, in particular, the probability matrix, from the data structure 130, and provide the first stage output, as an input, to the second stage OM 128. The intelligence engine 120 is configured to then, based on, among other things, the first stage output, execute the second stage OM 128 and, in particular, the plurality of object functions (e.g., as shown in Table 9, etc.), subject to the plurality of constraints (e.g., as shown in Table 10, etc.), to automatically generate an allocation plan 134 for the current test experiment without manual assignment by a user.

In connection therewith, the intelligence engine 120 is configured to execute the objective functions, subject to the plurality of constraints, of the second stage OM 128, based not only on the first stage output (e.g., the allocation prediction scores (e.g., Pᵢⱼ, etc.) as described above, etc.), but also based on the plurality of other indices and sets (as shown above in Table 6 above, etc.), decision variables (e.g., as shown above in Table 7, etc.), and/or function parameters (e.g., as shown above in Table 8, etc.). In this manner, the intelligence engine 120 is configured to, based on the execution of the second stage OM 128, generate a second stage output including the allocation plan 134 for the test experiment, where the allocation plan 134 takes into consideration not only historical allocation data as described above, but also considerations of maturity matching, environment, product segment distribution, and many other requirements at the same time.

The intelligence engine 120 is configured to then store the second stage output and, in particular, the allocation plan 134, in the data structure 130. Consistent with the above, the intelligence engine 120 is also configured to update the historical allocation data with the allocation plan 134, whereby the intelligence engine 120 is configured, for subsequent test experiments, to execute the first stage MLPM 126 based on the updated historical allocation data reflecting allocation plans for the current test experiment based on the test protocol data for the test protocols 112 of the current test experiment. In this manner, the intelligence engine 120 is configured as a self-learning system, whereby the engine 120 improves its intelligence on a continual basis as more allocation plans for more test experiments are generated (e.g., by retraining the recurrent neural network 132 and/or models 126 and 128 continually as more test experiments are generated, etc.).

In addition, the intelligence engine 120 may be configured, in one or more embodiments, to execute the second stage OM 128 and, in particular, one or more of the plurality of objective functions, based on one or more weights. In doing so, the intelligence engine 120 may be configured to receive the one or more weights as an input (e.g., via an input device 208 from a user or from another computing device, etc.). Alternatively, the intelligence engine may be configured to retrieve the one or more weights from the data structure 130. In one or more embodiments, the weights may be determined by domain experts, where the intelligence engine 120 is configured to execute the second stage MLPM 126 with one or more experimental weights. Feedback may then be obtained from the domain experts, and the weights may be adjusted accordingly based on the feedback. This process may be repeated for multiple interactions until appropriate and/or desired weights have been determined.

The intelligence engine 120 may also be configured, in one or more embodiments, to execute the second stage OM 128 and, in particular, one or more objective functions subject to one or more constraints, based on hub data. While the intelligence engine 120 permits test protocols 112 to be allocated to test locations 106 independent of the hubs (e.g., without requiring the test protocols 112 (and test sets 114) to be first allocated to the hubs for further advancement to the test locations 106), some of the constraints, for example (see, e.g., Table 10, etc.) are imposed as a hub level (e.g., constraints based on capacity of the hubs with which the test locations 106 are associated, etc.). As such, the hub data may include, for example, a set of hubs H (e.g., as shown above in Table 6, etc.), a capacity upper bound Cₕ^{U} of each hub h in the set of hubs H and/or a capacity lower bound Cₕ^{L} of each hub h in the set of hubs H, and/or a constraint that the total plots for each hub h should be between the capacity lower bound Cₕ^{L} of the hub h and the capacity upper bound Cₕ^{U} for the hub. In connection therewith, the intelligence engine 120 may be configured to receive the hub data from a file input to the intelligence engine 120 by a planting learn, etc.), from the data structure 130, or in one or more other manners.

That said, the allocation plan 134 generated as an output of the second stage OM 128 includes, for each test protocol 112 of the current test experiment, a test location 106 to which the test protocol 112 is to be allocated or advanced in the breeding pipeline for planting and testing, harvesting, etc. The intelligence engine 120 is then configured to store the allocation plan 134 in the data structure 130.

Table 11 illustrates an example allocation plan 134 generated by the second stage OM 128 when executed by the intelligence engine 120. In the example allocation plan 134, each test protocol 112 is identified by its ID (e.g., as defined in the test set characteristics, etc.) and each test location 106 is identified by its ID (e.g., as defined in the test location data, etc.). It should be appreciated that the allocation plan 134 may include additional information, different information, etc. in other embodiments.

TABLE 11

| Test Protocol ID | Test Location ID |
|---|---|
| P3057 | L1285 |
| | L9371 |
| | . . . |
| | L4327 |
| P3048 | L3019 |
| | L1285 |
| | . . . |
| | L9371 |

. . .

| | |
|---|---|
| P0381 | L4837 |
| | L9371 |
| | . . . |
| | L5281 |

The example allocation plan of Table 11 includes test protocol IDs for one through n test protocols 112, specifically: P3057 for a first test protocol 112, P3048 for a second test protocol 112, and P0381 for an n-th test protocol 112. The example allocation plan then also includes test location IDs for each of the test protocols and, in particular, for each test protocol 112, test locations IDs for the test locations 106 to which the test protocol 112 is to be allocated, whereby the multiple test sets 114 may be distributed to the multiple locations 106 (e.g., pursuant to the capacity reservation system 124, etc.).

With the allocation plan 134 generated, the intelligence engine 120 is configured to transmit the allocation plan 134 to the capacity reservation system 124 (e.g., in response a user instruction received via a user interface generated by the intelligence engine, etc.). In turn, the capacity reservation system 124 is configured to receive the allocation plan 134 and, based on the plan 134, to reserve space, resources, etc. at the test locations 106 to which the test protocols 112 have been allocated. Each test protocol 112 may then be advanced in the breeding pipeline to the test locations 106 to which the test protocol 112 has been allocated pursuant to the allocation plan 134 and for which space, resources, etc. have been reserved. When the test protocols 112 are advanced to their respective test locations 106, the test experiment may then be conducted on the test sets 114 of the corresponding test protocols 112. When completed, the test sets 114 and/or the corresponding test protocols 112 may then be advanced to a next stage of in the breeding pipeline (e.g., first screening stage, a second screening stage, a first pre-commercial stage, a second pre-commercial stage, a fourth, pre-commercial stage, a commercial stage, etc.). Although the intelligence engine 120 is described herein as generating allocation plans based on test protocols 112, in other embodiments the may generate allocation plans based on test sets 114, seeds 116 of the test sets 114, protocol data 118 for the test protocols 112, etc.

The intelligence engine 120 is also configured to generate a user interface based on the allocation plan 134. For instance, in response to a request from a user (e.g., for such user interface, etc.), the intelligence engine 120 is configured to retrieve the allocation plan 134 from the data structure 130 and generate the user interface based on the allocation plan 134 and the test location data, as well as protocol data for the test protocols 112 and hub capacity data. The user interface may provide an overview of the allocation plan 134, and also specific instructions as to physical labor at the test locations 106 in order to properly implement the allocation plan 134.

That said, whether based on a generated user interface, or otherwise, the breeding pipeline and test locations 106 (e.g., outdoor fields, indoor growing sites, etc.) included therein are physically conformed to the allocation plan 134. More specifically, the seeds 116 are planted consistent with the allocation plan 134 at the test locations 106. The seeds 116 may be planted manually or through automation, or via a combination thereof, and the resulting plants may be tested according to one or more suitable means, standards, protocols, etc. In this manner, the allocation plan 134 is physically implemented in the various test locations 106, whereby the seeds 116 (consistent with the test protocols 112) are populated across the various test locations 106 (and grown), whether the test locations 106 are associated with the same or different hubs.

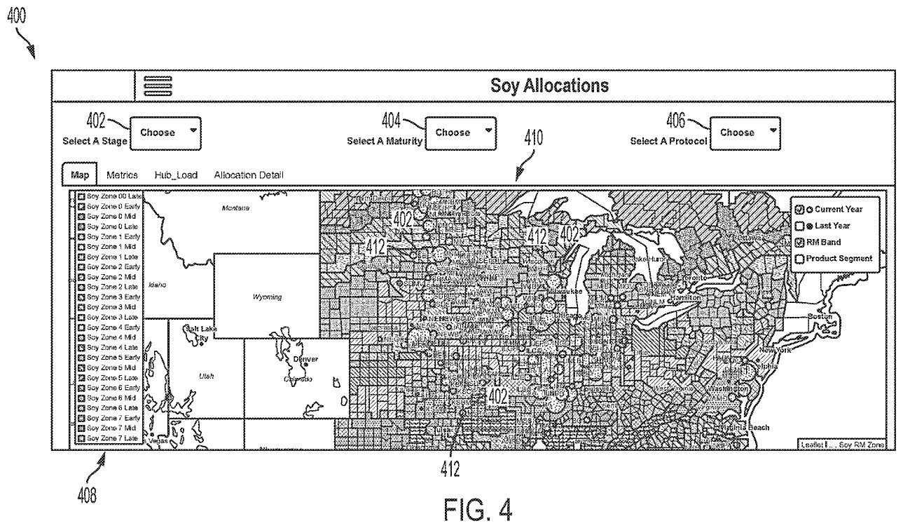

FIG. 4 illustrates an example interactive graphical user interface 400 generated by the intelligence engine 120. The user interface 400 generally includes a stage (e.g., crop material stage, etc.) selection field 402, a relative maturity (RM) selection field 404, and a test protocol selection field 406, a relative maturity (RM) key pane 408, and a map 410).

The stage selection field 402 is configured to allow a user to select one or more of a plurality of stages (e.g., crop material stages, etc.), such as a stage of development of interest for the seeds that are assigned to the test protocols 112 of interest and/or all stages of development of interest for plants/seeds that are permitted at the test locations 106, etc. Again, he selectable stages are not to be confused with the stages of the models 126 and 128. The RM selection field 404 is configured to allow a user to select one or more of a plurality of relative maturities of interest (e.g., RM of seeds 116 assigned to test protocols 112 of the interest and/or RM of plants/seeds planted or growing at the test locations 106 of the network 104, etc.). The RM key 408, then, is configured to, display a list of the RMs of interest selected by the user via the RM maturity selection field 404. In connection therewith, it should be appreciated that the various “Soy Zones” listed each correspond to one or more different relative maturities. For example, “Soy Zone 1 Early” corresponds to a relative maturity (RM) of (1.0, 1.3), “Soy Zone 1 Mid” corresponds to a relative maturity (RM) of (1.4, 1.6), and “Soy Zone 1 Late” corresponds to a relative maturity (RM) of (1.7, 1.9). And, the test protocol selection field 406 is configured to allow a user to select one or more of a plurality of test protocols 112 of interest for the given test experiment, where the plurality of selectable test protocols 112 are those that are included in the allocation plan 134 generated by the second stage OM 128.

The map 410 is configured illustrate the various regions 402 in which test locations 106 of the network 104 are geographically located. In connection therewith, the user interface 400 is configured, by the intelligence engine 120, to indicate the applicable RM for each corresponding test location 106 within each region 402, based on the indication keys provided in the RM key pane 408. The user interface 400 is then configured, by the intelligence engine 120 to, based on the user selection of the stages of interest and RMs of interest, identify the test locations 106 to which the test protocols 112 of interest have been assigned in the allocation plan 134. In the example user interface 400, such identification is via the allocation indicators 412.

The example user interface 400 further includes a “Metrics” tab, a “Hub_Load” tab, and an “Allocation Detail” tab. In connection therewith, the user interface 400 is configured, by the intelligence engine 120 to, based on a user selection of the “Metrics” tab, display key metrics attendant to the test experiment and/or the allocation plan 134 generated by the second stage OM 128 (e.g., RM matching between test protocols 112 and test locations 106, market size capture, etc.) (not shown). The user interface 400 is configured, by the intelligence engine 120 to, based on a user selection of the “Hub_Load” tab, display the percentage of hub capacity being used (e.g., for the hubs associated with the test locations 106 in the network 104, etc.) (not shown), whereby a user may track capacity for each hub. And, the user interface 400 is configured to, by the intelligence engine 120, based on a user selection of the “Allocation Detail” tab, display additional details for test protocol 112 allocated to test locations 106 and/or for the test locations 106 to which each test protocol 112 has been allocated (e.g., in table format, etc.)

In one or more embodiments, the user interface 400 may also be configured to, by the intelligence engine 120, receive a user selection/instruction to make a capacity reservation. In response to the selection/instruction, the user interface 400 may be configured to, by the intelligence engine 120, transmit the allocation plan 134 generated by the second stage OM 128 to the capacity reservation system 124, whereby the capacity reservation system 124 may reserve space, resources, etc. at the applicable test locations 106 as described above.

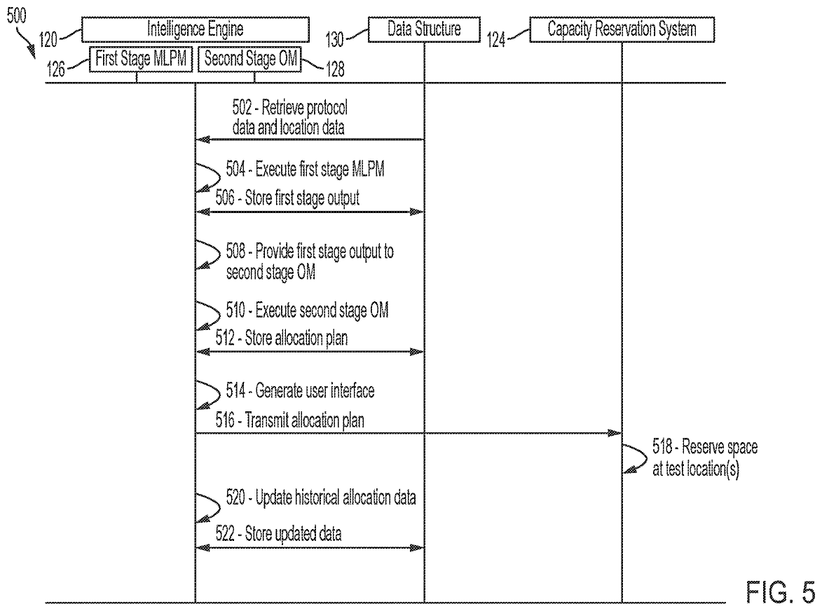

FIG. 5 illustrates an example method 500 for use in automatically allocating test protocols of a test experiment to test locations within a network of test locations. The example method 500 is described herein in connection with the intelligence engine 120 of the system 100, and is also described with reference to computing device 200. However, it should be appreciated that the methods herein are not limited to the system 100 (or the particular examples described therein), or the computing device 200. And, likewise, the systems and computing devices described herein are not limited to the example method 500.

Initially in the method 500, the intelligence engine 120 receives and/or retrieves (e.g., via an API, etc.), at 502, test protocol data for each test protocol 112 of a current test experiment from the data structure 130. For instance, the intelligence engine 120 may retrieve test requirements and test set characteristics for each test set 114 assigned to each test protocol 112 (e.g., the protocol data included in Tables 1 and 2, etc.). In addition, the intelligence engine 120 receives and/or retrieves, at 502, test location data for each of the test locations 106 within the network 104 (e.g., the data included in Table 3, etc.).

The intelligence engine 120 then executes the first stage MLPM 126 (trained as described above in the system 100), at 504, based on the retrieved test protocol data and test location data, to generate a first stage output. For example, executing the first stage MLPM 126 may include supplying the retrieved test requirements and test set characteristics for each test set 114 assigned to each test protocol 112 to the trained recurrent neural network 132 to generate the first stage output as an output of the recurrent neural network 132. And, at 506, the intelligence engine 120 stores the first stage output in the data structure 130. As generally described above, the first stage output includes, for each test location 106, an allocation preference score (broadly, an allocation prediction score) for each test protocol 112, in the form of a matrix. In connection therewith, again, each allocation prediction score represents a probability, based on the historical allocation data used to train the recurrent neural network 132 of the first stage MLPM 126, that the given test protocol 112 should be allocated or advanced to the corresponding test location 106.

Next in the method 500, the intelligence engine 120 provides the first stage output as an input to the second stage OM 126. In doing so, the intelligence engine 120 may provide the first stage output directly to the second stage OM 126. Or, the intelligence engine 120 may retrieve the first stage output from the data structure 130 (as stored at 506), and then provide the retrieved first stage output to the second stage OM 126. Regardless, using the first stage output, the intelligence engine 116 executes, at 510, the second stage OM 128 to generate an allocation plan 134 for the current test experiment. The allocation plan 134 includes, for each test protocol 112, a plurality of test locations 106 to which the test protocol 112 (and, in particular, the test sets 114 assigned to the test protocol 112) is to be allocated or advanced in the breeding pipeline for planting and/or testing and/or harvesting of the test sets 114, etc. For example, executing the second state OLM 128 may include executing a plurality of objective functions, subject to a plurality of constraints, based on a plurality of indices, sets, function parameters, decision variable, etc., to generate an optimized allocation plan 134. At 512, the intelligence engine 120 stores the allocation plan 134 in the data structure 130.

The intelligence engine 120 then generates, at 514, a user interface based on the allocation plan 134 (e.g., user interface 400, etc.) illustrating the test locations 106 for the test protocols 112. And, at 516, the intelligence engine 120 transmits the allocation plan 134 (e.g., via a network interface 210, etc.) to the capacity reservation system 124 (e.g., in response a selection by a user via the user interface 400, etc.). In turn, the capacity reservation system 124 receives (e.g., via a network interface 210) the allocation plan 134 and, at 518, based on the allocation plan 134, reserves space, resources, etc. at the test locations 106 to which the test protocols 112 have been allocated.

In this example embodiment, therefore, the breeding pipeline and test locations 106 (e.g., outdoor fields, indoor growing sites, etc.) included therein are physically conformed, again, to the allocation plan 134. More specifically, the seeds 116 are planted consistent with the allocation plan 134 at the test locations 106. The seeds 116 may be planted, manually or, through automation, or through a combination thereof, and the resulting plants (grown from the seeds) may be tested according to one or more suitable means. In this manner, the allocation plan 134 is physically implemented in the various test locations 106, whereby the seeds 116 (consistent with the test protocols 112) are populated across the various test locations 106 (and grown, cultivated, etc.), whether the test locations 106 are associated with the same or different hubs.

Finally in the method 500, the intelligence engine 120 updates, at 520, the historical allocation data to include the protocol data for each test protocol 112, the test location data for the test locations 106 in which the current test experiment is (or is to be) executed (e.g., based on test data from the seeds 116 planted in the test locations 106, etc.), and the allocation plan 134 generated as the output of the second stage OM 128. The intelligence engine 120 in turn stores, at 522, the updated historical allocation data in the data structure 130.

The updated historical allocation data may be used to re-train the recurrent neural network 132 and/or the MLPM 126 and the OM 128, to improve the automated allocation for future test protocols 112. Then, based on the updated historical allocation data, the intelligence engine 120 re-executes (consistent with steps 502 through 520) the first stage MLPM 126 and the second stage OM 128 with respect to a subsequent test experiment (different from the current test experiment), whereby an allocation plan 134 is generated for the subsequent test protocols 112 of the subsequent test experiment with the benefit of additional intelligence. This process may generally be repeated for any number of subsequent test experiments, whereby the intelligence of the engine 120 is continuously improved.

In view of the above, the systems and methods herein permit the automatic allocation of test protocols (for test sets of seeds assigned thereto or associated therewith) (substantially without human intervention) to test locations within a network of test locations, accounting for the number of test locations and the number of test protocols or test sets of the test experiment, regardless of the hub organization of the test sites within the network. What's more, the intelligence on which the automatic allocation is based is updated in an ongoing manner as allocations are performed, whereby subsequent allocations become more accurate, appropriate, and/or precise as time goes on. Automatically allocating test protocols to test location avoids the need for a user to manually evaluate the test locations on a continual basis, and then manually assign the protocols to locations based on the user evaluation of the test locations and the protocols. And, for allocation plans of the order described above (e.g., that include hundreds of test locations among various hubs, hundreds or thousands of test protocols, etc.), the manual human process of assigning test protocols to test locations is not feasible or even possible while satisfying all of the requirements and characteristics associated therewith. The intelligence engine 120 including the MLPM and OM allows historical allocation from multiple users to be combined into an automated allocation system, thereby improving the technology of test protocol and test location evaluation and allocation by optimizing the physical transportation and location of seeds of the test sets based on a complex set of characteristics and constraints. The intelligence engine 120 also improves the technology of seed test experiments by allocating a better range of distributions of seeds and test protocols across test locations having diverse properties for different test protocols, thereby increasing the success of the breeding pipeline experiments and outcomes.

The functions described herein, in some embodiments, may be described in computer executable instructions stored on a computer readable media, and executable by one or more processors. The computer readable media is a non-transitory computer readable media. By way of example, and not limitation, such computer readable media can include RAM, ROM, EEPROM, CD-ROM or other optical disk storage, magnetic disk storage or other magnetic storage device, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the following operations: (a) obtaining, by a computing device, a plurality of test protocols for a current test experiment, each test protocol corresponding to one or more test sets of seeds for the current test experiment; (b) executing, by a computing device, a first stage machine learning prediction model (MLPM) based on protocol data for a plurality of test protocols for a current test experiment to generate a first stage output, wherein the first stage MLPM is trained based on historical allocation data for one or more prior test experiments, wherein a plurality of test sets of seeds are associated with the plurality of test protocols, and wherein the first stage output includes, for a plurality of test locations, a plurality of allocation prediction scores for the plurality of test protocols; (c) based on the first stage output, executing, by the computing device, a second stage optimization model (OM) to generate a second stage output, wherein the second stage output includes an allocation plan for the plurality of test protocols, and wherein the allocation plan identifies one or more of the plurality of test locations for each of the plurality of test protocols; and (d) reserving one or more resources at the test location(s) identified by the allocation plan, for each of the plurality of test protocols.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. In addition, advantages and improvements that may be achieved with one or more example embodiments disclosed herein may provide all or none of the above mentioned advantages and improvements and still fall within the scope of the present disclosure.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on”, “engaged to”, “connected to”, “coupled to”, “associated with”, “in communication with”, or “included with” another element or layer, it may be directly on, engaged, connected or coupled to, or associated or in communication or included with the other feature, or intervening features may be present. As used herein, the term “and/or” and the phrase “at least one of” includes any and all combinations of one or more of the associated listed items.

Although the terms first, second, third, etc. may be used herein to describe various features, these features should not be limited by these terms. These terms may be only used to distinguish one feature from another. Terms such as “first”, “second”, and other numerical terms when used herein do not imply a sequence or order unless clearly indicated by the context. Thus, a first feature discussed herein could be termed a second feature without departing from the teachings of the example embodiments.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9, and so forth.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for use in allocating test protocols associated with a plant breeding pipeline to a plurality of test locations, the method comprising:
executing, by a computing device, a first stage machine learning prediction model (MLPM), based on protocol data for a plurality of test protocols associated with a plant breeding pipeline for a current test experiment, to generate a first stage output, wherein the first stage MLPM is trained based on historical allocation data for one or more prior test experiments, wherein a plurality of test sets of seeds are associated with the plurality of test protocols, and wherein the first stage output includes, for a plurality of test locations, a plurality of allocation prediction scores for the plurality of test protocols;
based on the first stage output, executing, by the computing device, a second stage optimization model (OM) to generate a second stage output, wherein the second stage output includes an allocation plan for the plurality of test protocols associated with the plant breeding pipeline, and wherein the allocation plan identifies one or more of the plurality of test locations for each of the plurality of test protocols; and
storing the second stage output in a memory, whereby the allocation plan is accessible to define planting, testing, and/or harvesting of the plurality of test sets of seeds in connection with the plant breeding pipeline.

2. The computer-implemented method of claim 1, wherein executing the first stage MLPM includes executing the first stage MLPM further based on test location data for the plurality of test locations; and
wherein the test location data identifies one or more characteristics of each test location.

3. The computer-implemented method of claims 1, further comprising:
generating, by the computing device, at least one interactive user interface representative of the allocation plan; and
displaying, by the computing device, the at least one interactive interface to a user in connection with planting the plurality of test sets of seeds.

4. The computer-implemented method of claim 1, further comprising:
planting the plurality of test sets of seeds associated with the plurality of test protocols in the plurality of test locations consistent with the allocation plan; and
harvesting plants from the plurality of test sets of seeds associated with the plurality of test protocols.

5. The computer-implemented method of claim 1, wherein the protocol data includes, for each test protocol: one or more requirements for the test protocol; and one or more characteristics for the test sets assigned to the test protocol; and/or
wherein the historical allocation data includes: one or more requirements for one or more historical test protocols; and one or more characteristics for test sets associated with the one or more historical test protocols.

6.-7. (canceled)

8. The computer-implemented method of claim 1, wherein the plurality of allocation prediction scores for the plurality of test sets represent probabilities that the test locations satisfy the test protocol data for the plurality of test protocols for the current test experiment; and/or
wherein the plurality of allocation prediction scores are included in a probability matrix, and wherein the probably matrix includes, for each of the plurality of test locations, an allocation prediction score for each of the plurality of test protocols of the current test experiment.

9. (canceled)

10. The computer-implemented method of claim 1, wherein the first stage MLPM includes a recurrent neural network trained based on the historical allocation data; and/or
wherein the second stage OM includes a plurality of multi-objective mixed-integer programming problems, and wherein executing the second stage OM includes executing the second stage OM subject to a plurality of constraints.

11. (canceled)

12. The computer-implemented method of claim 1, further comprising updating, by the computing device, the historical allocation data with test data based on plants grown from a plurality of seeds planted consistent with the allocation plan.

13. A system for use in allocating test protocols associated with a plant breeding pipeline to a plurality of test locations, the system comprising:
at least one processor configured to:
execute a first stage machine learning prediction model (MLPM), based on protocol data for a plurality of test protocols associated with a plant breeding pipeline for a current test experiment, to generate a first stage output, wherein the first stage MLPM is trained based on historical allocation data for one or more prior test experiments, wherein a plurality of test sets of seeds are associated with the plurality of test protocols, and wherein the first stage output includes, for a plurality of test locations, a plurality of allocation prediction scores for the plurality of test protocols;
based on the first stage output, execute a second stage optimization model (OM) to generate a second stage output, wherein the second stage output includes an allocation plan for the plurality of test protocols associated with the plant breeding pipeline, and wherein the allocation plan identifies one or more of the plurality of test locations for each of the plurality of test protocols; and
store the second stage output in a memory, whereby the allocation plan is accessible to define planting, testing, and/or harvesting of the plurality of test sets of seeds in connection with the plant breeding pipeline.

14. The system of claim 13, wherein the at least one processor is configured, in order to execute the first stage MLPM, to execute the first stage MLPM further based on test location data for the plurality of test locations.

15. The system of claim 13, wherein the protocol data includes, for each test protocol: one or more requirements for the test protocol; and one or more characteristics for the test sets assigned to the test protocol; and/or
wherein the test location data identifies one or more characteristics of each test location; and/or
wherein the historical allocation data includes: one or more requirements for one or more historical test protocols; and one or more characteristics for test sets associated with the one or more historical test protocols.

16.-17. (canceled)

18. The system of claim 13, wherein the plurality of allocation prediction scores for the plurality of test sets represent probabilities that the test locations satisfy the test protocol data for the plurality of test protocols for the current test experiment; and/or
wherein the plurality of allocation prediction scores are included in a probability matrix, and wherein the probably matrix includes, for each of the plurality of test locations, an allocation prediction score for each of the plurality of test protocols of the current test experiment.

19. (canceled)

20. The system of claim 13, wherein the first stage MLPM includes a recurrent neural network trained based on the historical allocation data; and/or
wherein the second stage OM includes a plurality of multi-objective mixed-integer programming problems, and wherein the at least one processor is configured, in order to execute the second stage OM, to execute the second stage OM subject to a plurality of constraints.

21.-23. (canceled)

24. The system of claim 13, wherein the at least one processor is further configured to:
direct the plurality of test sets of seeds associated with the plurality of test protocols to the plurality of test locations for planting, consistent with the allocation plan; and/or
direct plants from the plurality of test sets of seeds associated with the plurality of test protocols to be harvested and/or tested.

25. A non-transitory computer-readable storage medium including executable instructions which, when executed by at least one processor in connection with allocating test protocols associated with a plant breeding pipeline to a plurality of test locations, cause the at least one processor to:
execute a first stage machine learning prediction model (MLPM), based on protocol data for a plurality of test protocols associated with a plant breeding pipeline for a current test experiment, to generate a first stage output, wherein the first stage MLPM is trained based on historical allocation data for one or more prior test experiments, wherein a plurality of test sets of seeds are associated with the plurality of test protocols, and wherein the first stage output includes, for a plurality of test locations, a plurality of allocation prediction scores for the plurality of test protocols;
based on the first stage output, execute a second stage optimization model (OM) to generate a second stage output, wherein the second stage output includes an allocation plan for the plurality of test protocols associated with the plant breeding pipeline, and wherein the allocation plan identifies one or more of the plurality of test locations for each of the plurality of test protocols; and
store the second stage output in a memory, whereby the allocation plan is accessible to define planting, testing, and/or harvesting in connection with the plurality of test protocols associated with the plant breeding pipeline.

26. (canceled)

27. The non-transitory computer-readable storage medium of claim 25, wherein the protocol data includes, for each test protocol: one or more requirements for the test protocol; and one or more characteristics for the test sets assigned to the test protocol;
wherein the test location data identifies one or more characteristics of each test location; and
wherein the historical allocation data includes: one or more requirements for one or more historical test protocols; and one or more characteristics for test sets associated with the one or more historical test protocols.

28.-29. (canceled)

30. The non-transitory computer-readable storage medium of claim 25, wherein the plurality of allocation prediction scores for the plurality of test sets represent probabilities that the test locations satisfy the test protocol data for the plurality of test protocols for the current test experiment;
wherein the plurality of allocation prediction scores are included in a probability matrix; and
wherein the probably matrix includes, for each of the plurality of test locations, an allocation prediction score for each of the plurality of test protocols of the current test experiment.

31. (canceled)

32. The non-transitory computer-readable storage medium of claim 25, wherein the first stage MLPM includes a recurrent neural network trained based on the historical allocation data;
wherein the second stage OM includes a plurality of multi-objective mixed-integer programming problems; and
wherein the executable instructions, when executed by the at least one processor in order to execute the second stage OM, further cause the at least one processor to execute the second stage OM subject to a plurality of constraints.

33.-35. (canceled)

36. The non-transitory computer-readable storage medium of claim 25, wherein the executable instructions, when executed by the at least one processor, further cause the at least one processor to:
direct the plurality of test sets of seeds associated with the plurality of test protocols to the plurality of test locations for planting, consistent with the allocation plan; and/or
direct plants from the plurality of test sets of seeds associated with the plurality of test protocols to be harvested and/or tested.

37.-54. (canceled)

55. The computer-implemented method of claim 1, further comprising reserving one or more resources at the test location(s) identified by the allocation plan, for each of the plurality of test protocols.
